(19) United States
(12) Patent Application Publication
(10) Pub. No.: US 2025/0034062 A1
(43) Pub. Date: Jan. 30, 2025

(54) FIXED BED REACTORS AND PROCESSES FOR DEHYDRATION OF ALCOHOLS

(71) Applicant: Braskem S.A., Sao Paulo (BR)

(72) Inventors: Monica Albers VIEIRA, Sao Paulo (BR); Adler Gomes MOURA, Sao Paulo (BR); Andrea Marins OLIVEIRA, Sao Paulo (BR)

(21) Appl. No.: 18/771,535

(22) Filed: Jul. 12, 2024

Related U.S. Application Data
(60) Provisional application No. 63/526,754, filed on Jul. 14, 2023.

(51) Int. Cl. C07C 1/24; B01J 8/04

(52) U.S. Cl. CPC C07C 1/24; B01J 8/0492; B01J 8/0496; B01J 2208/00504; B01J 2208/00761; B01J 2208/021

(57) ABSTRACT

The disclosure provides a method of converting an alcohol into an olefin. The method includes passing a first fluid comprising the alcohol through a plurality of reactors. Each reactor includes a catalyst bed. During a catalyst campaign for at least one of the plurality of reactors, the corresponding catalyst bed includes a catalytic-active zone that is at least 90% of the catalyst bed. Additionally, the disclosure provides a system for converting an alcohol into an olefin.

### PRIORITY CLAIM

This application claims priority to U.S. Provisional Application No. 63/526,754, filed on Jul. 14, 2023, entitled “FIXED BED REACTORS AND PROCESSES FOR DEHYDRATION OF ALCOHOLS”, the entire disclosure of which is incorporated by reference herein.

### BACKGROUND

Ethylene may be produced by ethanol dehydration using fixed bed reactors. Despite providing good conversion and selectivity, reactors may not provide efficient catalyst usage due to layers in the catalyst bed that do not participate in the dehydration. These idle layers frequently operate at lower temperatures, leading to increased production of by-products such as ethane.

These idle layers also may lead to an increase in the pressure drop. The pressure drop may be closely related to the size of the compressor that follows the reaction and condensation sections. A higher pressure drop in the reactors may mean that the stream may be sent to the suction of the charge compressor at a lower pressure once the system is pressurized in the vaporization section and loses pressure along the equipment and pipes until it reaches the compressor. The suction pressure may be an important factor because the size of the compressor and the compression rate may be calculated based on this pressure once the discharge pressure is fixed, considering the same purification process. Therefore, a higher pressure drop may lead to a lower suction pressure, which may entail a higher number of stages for compression to achieve the pressure after the compression. A higher number of stages in the compression may result in higher capital and operational expenditures.

US 2013/0178674 A1 discloses a reactor design and configuration and a process for the catalytic dehydration of ethanol to ethylene.

WO 2021/247563 A1 discloses a trickle bed reactor including a plurality of catalyst beds connected in series and progressively increasing in catalyst mass in a direction from upstream to downstream.

There remains a need for smaller reactors by eliminating the idle layers and thus reducing the bed heights. There is also a need in the art to reduce consumption of catalyst, medium pressure steam, and/or power.

### SUMMARY

In one aspect, the present disclosure provides a method of converting an alcohol into an olefin. The method includes passing a first fluid including the alcohol through a plurality of reactors, wherein each reactor may include a catalyst bed. During a catalyst campaign for at least one of the plurality of reactors, the corresponding catalyst bed includes a catalytic-active zone that is at least 90% of the catalyst bed. In one embodiment, the catalytic-active zone may be 100% of the catalyst bed during the catalyst campaign. In one embodiment, at least one of the catalyst beds may further include an idle catalyst zone that may be less than 10% of the catalyst bed during the catalyst campaign. In one embodiment, at least one of the catalyst beds may not include an idle catalyst zone during the catalyst campaign. In one embodiment, the catalytic-active zone may operate at a temperature of 280° C. to 500° C. In one embodiment, the catalytic-active zone may operate at a temperature of 360° C. to 470° C. In one embodiment, the alcohol may include at least one of a C₂ alcohol, a C₃ alcohol, and a C₄ alcohol. In one embodiment, the olefin may include an alkene having the same number of carbons atoms as the alcohol. In one embodiment, the olefin may include ethylene, and the catalytic-active zone may operate at a temperature of 360° C. to 470° C.

In one embodiment, the method may further include contacting the first fluid with a second fluid comprising steam prior to the first fluid contacting the plurality of reactors, wherein a ratio of the steam to the alcohol may be from 1:1 to 3:1, obtaining a third fluid from at least one of the plurality of reactors, wherein the third fluid comprises the alcohol at a concentration lower than a concentration of the alcohol in the first fluid, contacting the third fluid with the first fluid prior to contacting the third fluid with another reactor among the plurality of reactors, and obtaining from an end of the plurality of reactors, a fourth fluid comprising the olefin and an alkane that has the same number of carbon atoms. In one embodiment, an amount of the olefin in the fourth fluid may be at least 1,000 ppm, mole basis, greater than an amount of the alkane. In one embodiment, an amount of the alkane in the fourth fluid may be 2,500 ppm or less.

In one embodiment, the method may further include pressuring the fourth fluid with a one-stage compressor connected to an end of the plurality of reactors. In one embodiment, the method may exclude pressurizing the fourth fluid with a multi-stage compressor.

In one embodiment, the method may further include feeding the first fluid to at least one of the plurality of reactors at a temperature more than 280° C.

In one embodiment, the plurality of reactors may be in a series-parallel arrangement. In one embodiment, at least one of the plurality of reactors has a weight hourly space velocity of the alcohol of more than 0.6/h and up to 1.5/h. In one embodiment, at least one of the plurality of reactors has a weight hourly space velocity of the alcohol of more than 1/h and up to 1.3/h.

In one embodiment, the plurality of reactors may include a first group of reactors including reactors each having a first height, and a second group of reactors including reactors each having a second height, where the first height may be different from the second height. In one embodiment, the plurality of reactors may include, in order, a first reactor including a first catalyst bed, a second reactor including a second catalyst bed, a third reactor including a third catalyst bed, and a fourth reactor including a fourth catalyst bed. In one embodiment, the plurality of reactors further may include, after the fourth reactor, a fifth reactor including a fifth catalyst bed. In one embodiment, a variance (difference) between a height of each of the plurality of reactors may be no more than 15%. In one embodiment, a variance (difference) between a height of each of the plurality of reactors may be no more than 10%. In one embodiment, each of the plurality of reactors independently may have a height of from 2.5 m to 4.0 m. In one embodiment, the first reactor may have a height of 2.8 to 3.1 m. In one embodiment, the third reactor may have a height of 3.2 to 3.5 m. In one embodiment, the first and second reactors may have the same height. In one embodiment, the remaining reactors may have a height different from the first and second reactors. In one embodiment, each of the third and fourth reactors may have a height that is 10% to 50% greater than the height of the first reactor. In one embodiment, the fifth reactor may have a height that is 10% to 50% greater than the height of the first reactor. In one embodiment, the third to fifth reactors may have the same height. In one embodiment, the fourth and fifth reactors may have the same volume. In one embodiment, a volume of each of the first to fourth reactors may increase from the first reactor to the fourth reactor.

In one embodiment, a variance (difference) between a height of each of the catalyst beds may be no more than 15%. In one embodiment, each of the catalyst beds independently may have a height of from 2.0 m to 2.8 m. In one embodiment, the first catalyst bed may have a height of 2.0 m to 2.2 m. In one embodiment, the third catalyst bed may have a height of 2.3 m to 2.6 m. In one embodiment, the first and second catalyst beds may have the same height. In one embodiment, the third and fourth catalyst beds may have a height different from the first and second catalyst bed. In one embodiment, each of the third and fourth catalyst beds may have a height that is 10% to 50% greater than the height of the first catalyst bed. In one embodiment, the fifth catalyst bed may have a height that is 10% to 50% greater than the height of the first catalyst bed. In one embodiment, the fourth and fifth catalyst beds may have the same height. In one embodiment, the fourth and fifth catalyst beds may have the same volume. In one embodiment, a volume of the catalyst beds may increase from the first catalyst bed to the fourth catalyst bed.

In one embodiment, at least one of the plurality of reactors may be fluidly coupled to a furnace to form a furnace-reactor couple. In one embodiment, the method may further include passing the first fluid through the furnace before the first fluid enters the reactor in the same furnace-reactor couple. In one embodiment, the furnace-reactor couple may include a single furnace and a single reactor. In one embodiment, a plurality of the furnace-reactor couples may be present, and each of the furnace-reactor couples may be fluidly coupled to each other. In one embodiment, at least one of the plurality of the furnace-reactor couples may be a spare furnace-reactor couple. In one embodiment, the spare furnace-reactor couple may include a reactor that may have the same height and/or volume as another reactor in another furnace-reactor couple among the plurality of the furnace-reactor couples. In one embodiment, the spare furnace-reactor couple may include a reactor that may have the same height and/or volume as another reactor in another furnace-reactor couple immediately preceding the spare furnace-reactor couple. In one embodiment, the plurality of the furnace-reactor couples may include five furnace-reactor couples. In one embodiment, only one of the five of the furnace-reactor couples may be a spare furnace-reactor couple. In one embodiment, the spare furnace-reactor couple may be configured to be a spare for any of the other furnace-reactor couples among the plurality of furnace-reactor couples. In one embodiment, the spare furnace-reactor couple may be the last furnace-reactor couple among the plurality of the furnace-reactor couples. In one embodiment, the reactor in the spare furnace-reactor couple may have the greatest height and/or volume among reactors in the plurality of the furnace-reactor couples. In one embodiment, all of the plurality of the furnace-reactor couples may operate together at 60% to 80% of capacity.

In one embodiment, the catalyst campaign may be between five and seven months. In one embodiment, the catalyst campaign may be six months.

In one embodiment, the method may further include, after the catalyst campaign, decoupling at least one of the plurality of the furnace-reactor couples from the remaining furnace-reactor couples, and regenerating the catalyst bed or changing the catalyst bed in the at least one decoupled furnace-reactor couple. In one embodiment, the decoupling may occur immediately after the catalyst campaign. In one embodiment, the remaining furnace-reactor couples may operate together at 100% of capacity.

In one aspect, the present disclosure provides a system including a plurality of reactors, wherein each reactor may include a catalyst bed configured to convert an alcohol into an olefin. The plurality of reactors is fluidly coupled to each other, and at least one of the catalyst beds is configured to have a catalytic-active zone that is at least 90% of the catalyst bed during a catalyst campaign. In one embodiment, the catalytic-active zone may be 100% of the catalyst bed during the catalyst campaign. In one embodiment, at least one of the catalyst beds may further include an idle catalyst zone that is less than 10% of the catalyst bed during the catalyst campaign. In one embodiment, at least one of the catalyst beds may not include an idle catalyst zone during the catalyst campaign. In one embodiment, the alcohol may include at least one of a C₂ alcohol, a C₃ alcohol, and a C₄ alcohol. In one embodiment, the olefin may include ethylene.

In one embodiment, the plurality of reactors may be in a series-parallel arrangement. In one embodiment, at least one of the plurality of reactors has a weight hourly space velocity of the alcohol of more than 0.6/h and up to 1.5/h. In one embodiment, at least one of the plurality of reactors has a weight hourly space velocity of the alcohol of more than 1/h and up to 1.3/h.

In one embodiment, the plurality of reactors may include a first group of reactors including reactors each having a first height, and a second group of reactors including reactors each having a second height, where the first height may be different from the second height. In one embodiment, the plurality of reactors may include, in order, a first reactor including a first catalyst bed, a second reactor including a second catalyst bed, a third reactor including a third catalyst bed, and a fourth reactor including a fourth catalyst bed. In one embodiment, the plurality of reactors further may include, after the fourth reactor, a fifth reactor including a fifth catalyst bed. In one embodiment, a variance (difference) between a height of each of the plurality of reactors may be no more than 15%. In one embodiment, a variance (difference) between a height of each of the plurality of reactors may be no more than 10%. In one embodiment, each of the third and fourth reactors may have a height that is 10% to 50% greater than the height of the first reactor. In one embodiment, the fifth reactor may have a height that is 10% to 50% greater than the height of the first reactor. In one embodiment, the third to fifth reactors may have the same height. In one embodiment, the fourth and fifth reactors may have the same volume. In one embodiment, a volume of each of the first to fourth reactors may increase from the first reactor to the fourth reactor.

In one embodiment, a variance (difference) between a height of each of the catalyst beds may be no more than 15%. In one embodiment, each of the third and fourth catalyst beds may have a height that is 10% to 50% greater than the height of the first catalyst bed. In one embodiment, the fifth catalyst bed may have a height that is 10% to 50% greater than the height of the first catalyst bed. In one embodiment, the fourth and fifth catalyst beds may have the same height. In one embodiment, the fourth and fifth catalyst beds may have the same volume. In one embodiment, a volume of the catalyst beds may increase from the first catalyst bed to the fourth catalyst bed.

In one embodiment, at least one of the plurality of reactors may be fluidly coupled to a furnace to form a furnace-reactor couple. In one embodiment, a plurality of the furnace-reactor couples may be present, and each of the furnace-reactor couples may be fluidly coupled to each other. In one embodiment, at least one of the plurality of the furnace-reactor couples may be a spare furnace-reactor couple. In one embodiment, the spare furnace-reactor couple may include a reactor that may have the same height and/or volume as another reactor in another furnace-reactor couple among the plurality of the furnace-reactor couples. In one embodiment, the spare furnace-reactor couple may include a reactor that may have the same height and/or volume as another reactor in another furnace-reactor couple immediately preceding the spare furnace-reactor couple. In one embodiment, the plurality of the furnace-reactor couples may include five furnace-reactor couples. In one embodiment, only one of the five of the furnace-reactor couples may be a spare furnace-reactor couple. In one embodiment, the spare furnace-reactor couple may be configured to be a spare for any of the other furnace-reactor couples among the plurality of furnace-reactor couples. In one embodiment, the spare furnace-reactor couple may be the last furnace-reactor couple among the plurality of the furnace-reactor couples. In one embodiment, the reactor in the spare furnace-reactor couple may have the greatest height and/or volume among reactors in the plurality of the furnace-reactor couples.

### BRIEF DESCRIPTION OF THE DRAWINGS

The foregoing summary, as well as the following detailed description of the disclosure, will be better understood when read in conjunction with the appended figures. It should be understood, however, that the disclosure is not limited to the precise arrangements, examples and instrumentalities shown in the figures.

FIG. 1 is a diagram illustrating the reaction front moving downwards as the reaction progresses.

FIG. 2 is a diagram illustrating the minimum reaction front section and buffer section in a catalytic bed in a reactor in a comparative process.

FIG. 3 is a diagram illustrating the catalyst usage along the bed (left) and catalyst selectivity in the layers (right). FIG. 3 illustrates how the bottom layers of catalyst, particularly, the idle catalyst, may be unused, as well as the selectivity in each layer.

FIG. 4 is a diagram illustrating the furnace-reactor couples in the process according to an embodiment of the present disclosure.

FIG. 5 is scheme illustrating the arrangement of reactors used in an ethanol dehydration process in a comparative process.

FIG. 6 is a diagram illustrating the temperature profile change over time.

FIG. 7 is a chart showing the comparison between pressure curves in the comparative process and the process according to an embodiment of the present disclosure.

FIG. 8 is a chart showing the change in ethane concentration (ppm) and yield (%) during a catalyst campaign.

### DETAILED DESCRIPTION

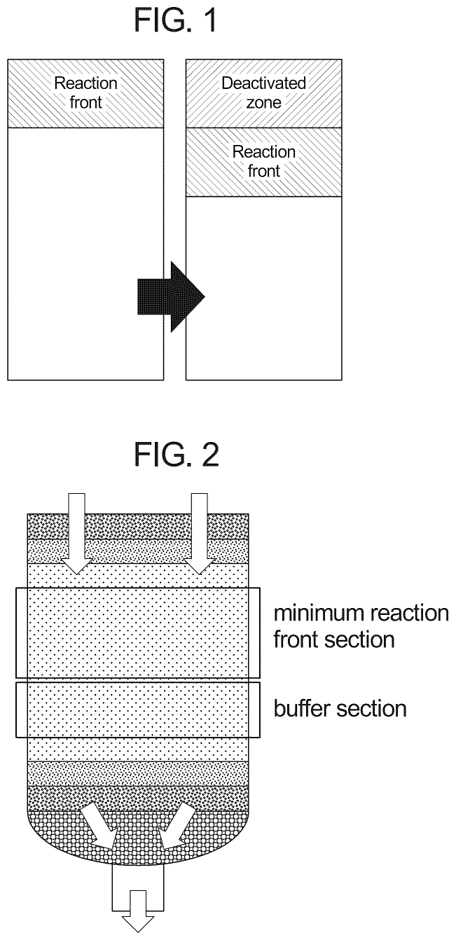

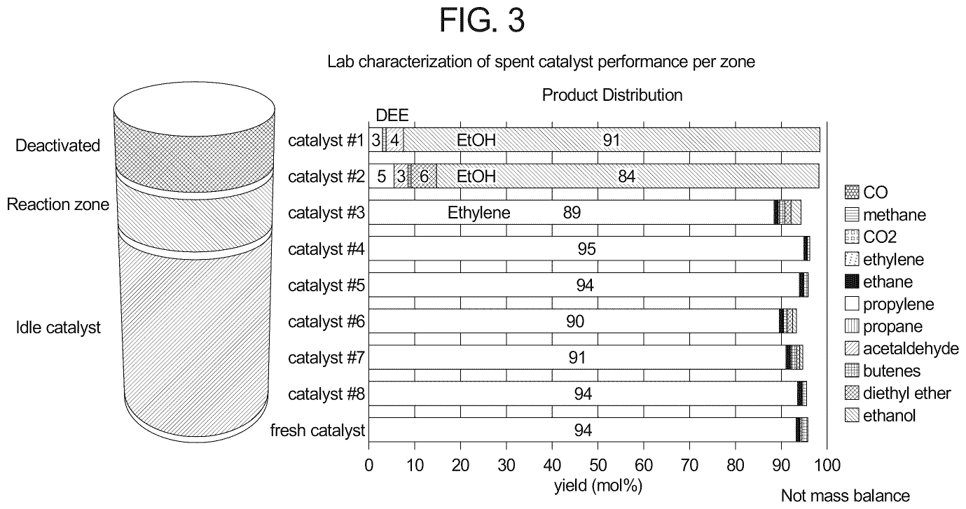

The present disclosure provides a method of converting an alcohol into an olefin using smaller reactors by reducing the amount of idle catalyst layers and thus reducing the catalyst bed height. Without wishing to be bound by theory, it is believed that reducing the catalyst bed height improves selectivity due to the lower by-product formation, and also reduces pressure drop along the catalyst bed, which is believed to facilitate a smaller compressor size. To calculate the catalyst bed height, a relevant concept is the reaction front section. The reaction front is defined as the location along the catalyst bed where most of the chemical reaction occurs in a given moment. As the reaction progresses, the reaction front slowly moves downwards, as illustrated in FIG. 1. Theoretically, the reaction front should reach the bottom of the reactor at the end of the catalyst campaign. After that, the catalyst will be regenerated, and the new catalyst campaign begins.

FIG. 2 shows the minimum reaction front section of the catalytic bed. The buffer section is the distance available for the reaction front to travel through over the catalyst campaign. For the reactor design in a comparative process having idle layers, the reaction front may never reach the bottom of the reactor. The reaction front may move only up to half the length of the catalytic bed. The reason for this may be that by-product formation increases so much that catalyst regeneration may be required before the reaction front reaches the bottom. One possible reason for the by-product formation may be the temperature (e.g., temperature lower than the dehydration temperature) in the buffer section. Early catalyst regeneration may indicate that the bed height is overdesigned. An overdesigned reactor may provide a buffer section longer than it should be and, therefore, by-product formation is higher than it could be. Long bed heights may lead to low temperatures in the idle catalyst layers in the buffer section, which may lead to unnecessary by product formation. The overall yield and selectivity may be reduced. The desired bed height may be longer than the minimum reaction front section but shorter than the bed height of reactors in the comparative process.

In one embodiment, the smaller reactors may operate with the same amount of layers that may be used in the reaction front in comparative systems, and may reduce the problem with by-products. This feature may increase the selectivity and may reduce the pressure drop in each reactor, making the use of a smaller charge compressor practically feasible. In one embodiment, the disclosed methods for the dehydration of alcohol and the industrially important product(s) may be cost efficient and/or energy efficient. In one embodiment, the methods for the dehydration of alcohol may be made cost efficient by using smaller reactors and reducing catalyst consumption. In one embodiment, the methods for separation may be made energy efficient through reducing power consumption by using a single-stage compressor instead of a multi-stage compressor (e.g., a three-stage compressor). The present disclosure also provides a system for converting the alcohol into the olefin.

As used herein, the term “catalyst campaign” is intended to refer to a period when a particular reactor is in operation. During the operation, the alcohol is dehydrated to form the olefin. “Catalyst campaign” refers to the period between catalyst regeneration and excludes the period when the spent catalyst is regenerated.

As used herein, the term “catalyst bed” is intended to refer the catalyst layers inside a single reactor and composed by the catalyst itself excluding the inerts.

As used herein, the term “catalytic-active zone” is intended to mean a region in which the catalysis of the dehydration of alcohol occurs in the catalytic bed at any time during the catalyst campaign.

As used herein, the term “idle catalyst zone” is intended to mean a region in which the catalytic dehydration of the alcohol never occurs in the catalytic bed during the catalyst campaign.

The boundary separating the catalytic-active zone and the idle catalyst zone during the catalyst campaign may be determined by the temperature measured by, for example, temperature transmitters in direct thermal contact with the catalyst bed.

As used herein, the term “catalyst” is intended to mean catalysts suitable for dehydration of alcohol as understood by one of ordinary skill in the art, including, but not limited to, alumina (e.g., γ-Al₂O₃), silica-alumina, zeolites (e.g., HZSM-5), metal oxide, or supported phosphoric acid and phosphate. The catalyst may be in a solid state (e.g., pellet, powder or particle).

As used herein, the term “catalyst cycle” is intended to refer to the lifespan of a catalyst inside the reactor and from a new catalyst state until the replacement of the catalyst.

As used herein, the term “C₂ alcohol” is intended to mean ethanol with a general formula CH₃CH₂OH (CAS number: 64-17-5).

As used herein, the term “C₃ alcohol” is intended to cover n-propanol with a general formula CH₃CH₂CH₂OH (CAS number: 71-23-8) and/or isopropyl alcohol with a general formula (CH₃)₂CHOH (CAS number: 67-63-0).

As used herein, the term “C₄ alcohol” is intended to cover n-butanol with a general formula CH₃CH₂CH₂CH₂OH (CAS number: 71-36-3), sec-butyl alcohol with a general formula CH₃CH₂CH(OH)CH₃ (CAS number: 78-92-2), isobutyl alcohol with a general formula CH₃CH(CH₃)CH₂OH (CAS number: 78-83-1), and tert-butyl alcohol with a general formula (CH₃)₃COH (CAS number: 75-65-0).

As used herein, the term “C₂ alkene” is intended to mean ethylene with a general formula CH₂═CH₂ (CAS number: 74-85-1).

As used herein, the term “C₃ alkene” is intended to cover propene with a general formula CH₂═CHCH₃ (CAS number: 115-07-1).

As used herein, the term “C₄ alkene” is intended to cover 1-butene with a general formula CH₃CH₂CH—CH₂ (CAS number: 106-98-9), cis-2-butene with a general formula CH₃CH═CHCH₃ (CAS number: 590-18-1), trans-2-butene with a general formula CH₃CH═CHCH₃ (CAS number: 624-64-6), and isobutylene with a general formula (CH₃)₂C═CH₂ (CAS number: 115-11-7).

As used herein, the term “weight hourly space velocity” or “WHSV” is intended to mean the specific mass flow rate of alcohol per mass (e.g., kg) of catalyst in the catalyst bed. The specific mass flow rate of alcohol may be measured by methods known to one of ordinary skill in the art.

As used herein, the term “regenerating the catalyst bed” is intended to mean a process to restore the catalytic activity of a spent catalyst.

As used herein, the term “changing the catalyst bed” is intended to mean a process to replaces a spent catalyst with a fresh catalyst.

As used herein, the term “capacity” of the furnace-reactor couple is intended to mean Vo/V, where Vo represents the massic flow rate of alcohol entering the reactor and V represents the volume of the reactor itself.

Unless defined otherwise herein, all technical and scientific terms used herein have the same meaning as commonly understood by one of ordinary skill in the art to which this disclosure belongs. Jonathan Law et. al., Oxford Dictionary of Chemistry, Oxford University Press, Oxford (2020) and Carl Schaschke, Oxford Dictionary of Chemical Engineering, Oxford University Press, Oxford (2014) provide one of skill with a general dictionary of many of the terms used in this disclosure.

While the present disclosure is capable of being embodied in various forms, the description below of several embodiments is made with the understanding that the present disclosure is to be considered as an exemplification of the disclosure, and is not intended to limit the disclosure to the specific embodiments illustrated. Headings are provided for convenience only and are not to be construed to limit the disclosure in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Use of the term “about” or “approximately” when referring to a number or a numerical range means that the number or numerical range referred to is an approximation within experimental variability (or within statistical experimental error), and thus the number or numerical range may vary from, for example, between 1% and 15% of the stated number or numerical range. Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Numeric ranges provided herein are inclusive of the numbers defining the range. Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Method of Converting an Alcohol into an Olefin

In one aspect, the present disclosure provides a method of converting an alcohol into an olefin. The method includes passing a first fluid including the alcohol through a plurality of reactors. Each reactor may include a catalyst bed. During a catalyst campaign for at least one of the plurality of reactors, the corresponding catalyst bed includes a catalytic-active zone that is at least 90% of the catalyst bed. In some embodiments, the corresponding catalyst bed may include a catalytic-active zone that is at least 91%, 92%, 93%, 94%, 95%, 96%, 97%, 98%, or 99% of the catalyst bed. In one embodiment, the catalytic-active zone may be 100% of the catalyst bed during the catalyst campaign. The proportion of the catalytic-active zone relative to the catalyst bed may be based on a height of the catalytic-active zone during the catalyst campaign and the height of the entire catalyst bed in the reactor. The height of the catalytic-active zone may be determined as described further below. In some embodiments, the proportion of the catalytic-active zone relative to the catalyst bed may be based on a volume of the catalytic-active zone during the catalyst campaign and the volume of the entire catalyst bed in the reactor. The volume of the catalytic-active zone may be calculated using the formula, πr²h, where r is the radius of the catalyst bed and h is the height of the catalytic-active zone.

In one embodiment, the catalytic-active zone may operate at a temperature of 280° C. to 500° C. during the catalyst campaign. In some embodiments, the boundary of the catalytic-active zone may be at least 280° C., 300° C., 320° C., 340° C., 360° C., 380° C., 400° C., 420° C., 440° C., 460° C., or 480° C., and/or less than 300° C., 320° C., 340° C., 360° C., 380° C., 400° C., 420° C., 440° C., 460° C., 480° C. or 500° C., or the temperature at which catalytic dehydration of the alcohol to the olefin occurs. The height of the catalytic-active zone may be the vertical distance between the boundary of the catalytic-active zone and the topmost surface of the catalyst bed. In some embodiments, the catalytic-active zone may operate at a temperature of at least 280° C., 300° C., 320° C., 340° C., 360° C., 380° C., 400° C., 420° C., 440° C., 460° C. or 480° C., and/or up to 300° C., 320° C., 340° C., 360° C., 380° C., 400° C., 420° C., 440° C., 460° C., 480° C. or 500° C. In one embodiment, the catalytic-active zone may operate at a temperature of 360° C. to 470° C.

In one embodiment, the olefin comprises ethylene, and the catalytic-active zone operates at a temperature of 360° C. to 470° C.

The temperatures may be measured by, for example, temperature transmitters in direct thermal contact with the catalyst bed. The temperature transmitters may be disposed along the height of the reactor. A distance between the transmitters may be at least 0.2 m, 0.3 m, 0.4 m, 0.5 m, or 0.6 m and/or not more than 0.4 m, 0.5 m, 0.6 m, 0.7 m, 0.8 m, 0.9 m or 1 m. Depending on the height of the reactor, the distance between the transmitters may be at least 0.8 m, 0.81 m, 0.82 m, 0.83 m, 0.85 m, 0.86 m, 0.87 m, 0.88 m, 0.89 m, 0.9 m, 0.91 m, 0.92 m, 0.93 m, and/or not more than 0.82 m, 0.83 m, 0.85 m, 0.86 m, 0.87 m, 0.88 m, 0.89 m, 0.9 m, 0.91 m, 0.92 m, 0.93 m, 0.94 m, 0.95 m, 0.96 m, 0.97 m or 0.98 m.

In one embodiment, at least one of the catalyst beds may further include an idle catalyst zone that may be less than 10% of the catalyst bed during the catalyst campaign. In some embodiments, the idle catalyst zone may be less than 9%, 8%, 7%, 6%, 5%, 4%, 3%, 2%, or 1% of the catalyst bed during the catalyst campaign. In one embodiment, at least one of the catalyst beds may not include an idle catalyst zone during the catalyst campaign. A temperature of the idle catalyst zone may be lower than 280° C., 300° C., 320° C., 340° C., 360° C., 380° C., 400° C., 420° C., 440° C., 460° C., 480° C., or the lower temperature limit at which catalytic dehydration of the alcohol to the olefin occurs. The proportion of the idle catalyst zone relative to the catalyst bed may be based on a height of the idle catalyst zone during the catalyst campaign and the height of the entire catalyst bed in the reactor. The height of the idle catalyst zone may be the vertical distance between the boundary of the catalytic-active zone and the bottommost surface of the catalyst bed.

In some embodiments, the first fluid may be fed to at least two, three, four, five, six, seven, eight or nine reactors among the plurality of reactors. In some embodiments, the first fluid may be fed to each reactor. In some embodiments, the first fluid may consist of the alcohol. In some embodiments, the first fluid may include the alcohol as the main component.

In one embodiment, the method may further include feeding the first fluid to at least one of the plurality of reactors at a temperature more than 280° C. The temperature may be measured by, for example, temperature transmitter(s) disposed at the inlet of each reactor or at the inlet of each furnace in the furnace-reactor couple. In some embodiments, the temperature may be more than 300° C., 320° C., 340° C., 360° C., 380° C., 400° C., 420° C., 440° C., 460° C. or 480° C. In one embodiment, the temperature may be more than 400° C. In one embodiment, the temperature may be more than 450° C. In one embodiment, the temperature may be more than 400° C. In one embodiment, the temperature may be more than 450° C. In some embodiments, the first fluid includes ethanol, which may be fed to the reactors at 470° C.

In one embodiment, the method may further include contacting the first fluid with a second fluid including steam prior to the first fluid contacting the plurality of reactors, wherein a ratio of the steam to the alcohol is from 1:1 to 3:1, obtaining a third fluid from at least one of the plurality of reactors, wherein the third fluid comprises the alcohol at a concentration lower than a concentration of the alcohol in the first fluid, contacting the third fluid with the first fluid prior to contacting the third fluid with another reactor among the plurality of reactors, and obtaining from an end of the plurality of reactors, a fourth fluid comprising the olefin and an alkane that has the same number of carbon atoms.

In one embodiment, the first fluid may contact the second fluid prior to the first fluid contacting the first reactor. In one embodiment, the first fluid may contact the second fluid prior to the first fluid contacting the second reactor. In one embodiment, the first fluid may contact the second fluid prior to the first fluid contacting each of the first and second reactor.

In some embodiments, the second fluid may consist of steam. In some embodiments, the second fluid may include steam as the main component. In one embodiment, the steam may be medium pressure steam, as understood by one skilled in the art. The medium pressure steam may have a pressure of 14 kgf/cm². In some embodiments, a ratio of the steam to the alcohol may be at least 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, or 2.9:1, and/or up to 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, or 3:1. In some embodiments, the ratio may be 2.5:1. In some embodiments, the ratio of the steam to the alcohol may refer to a ratio of the feed flow rates of steam and alcohol. The feed flow rate may be a mass flow rate.

In some embodiments, the third fluid may include the alcohol and the olefin. In some embodiments, the third fluid may be fed to the immediately following reactor. In some embodiments, the third fluid may be fed to only the immediately following reactor. For example, the third fluid from the penultimate reactor may be fed to only the last reactor. In some embodiments, the third fluid may be fed to the immediately following reactor in operation For example, the third fluid from the first reactor may be fed to the second or third reactors. For example, the third fluid from the second reactor may be fed to the third or fourth reactors. For example, the third fluid from the third reactor may be fed to the fourth or fifth reactors. For example, the third fluid from the fourth reactor may be fed to the fifth or to the quench tower.

In some embodiments, the third fluid may contact the first fluid before contacting the immediately following reactor. In some embodiments, the third fluid may contact the first fluid before contacting the two, three, four, five, six, seven or eight immediately following reactors.

In some embodiments, the fourth fluid may be obtained from the last reactor among the plurality of reactors. In some embodiments, the fourth fluid may be obtained from both the last and penultimate reactors among the plurality of reactors. In some embodiments, the fourth fluid may be obtained from only the last and penultimate reactors among the plurality of reactors.

In one embodiment, an amount of the olefin in the fourth fluid is at least 1,000 ppm, mole basis, greater than an amount of the alkane. In some embodiments, an amount of the olefin in the fourth fluid may be at least 1,000 ppm, 2,000 ppm, 3,000 ppm, 4,000 ppm, 5,000 ppm, 10,000 ppm, 15,000 ppm, or 20,000 ppm, mole basis, greater than an amount of the alkane.

In one embodiment, an amount of the alkane in the fourth fluid may be 2,500 ppm or less. In some embodiments, an amount of the alkane in the fourth fluid may be 2,400 ppm or less, 2,200 ppm or less, 2,000 ppm or less, 1,800 ppm or less, 1,500 ppm or less, 1,200 ppm or less, 1,000 ppm or less, 800 ppm or less, or 500 ppm or less. In some embodiments, the fourth fluid may be free of the alkane. The amount of alkane and olefin may be measured by gas chromatography or any other methods known to one of ordinary skill in the art.

In some embodiments, a conversion of the alcohol into the olefin may be at least 80%, 81%, 82%, 83%, 84%, 85%, 86%, 87%, 88%, 89%, 90%, 91%, 92%, 93%, 94%, 95%, 96%, 97%, 98% or 99%, based on the number of moles of alcohol.

In one embodiment, the method may further include pressuring the fourth fluid with a one-stage compressor connected to an end of the plurality of reactors. For example, the one-stage compressor may be connected to the last reactor. In some embodiments, the one-stage compressor may be a single-stage compressor. In one embodiment, the method may exclude pressurizing the fourth fluid with a multi-stage compressor.

### Catalyst Bed

In some embodiments, a catalyst bed may include at least 80 wt %, 81 wt %, 82 wt %, 83 wt %, 84 wt %, 85 wt %, 86 wt %, 87 wt %, 88 wt %, 89 wt %, 90 wt %, 90 wt %, 91 wt %, 92 wt %, 93 wt %, 94 wt %, 95 wt %, 96 wt %, 97 wt %, 98 wt % or 99 wt % of catalyst based on a total weight of the components in the catalyst bed. In some embodiments, the catalyst bed may include 100 wt % of catalyst.

In some embodiments, there may be three, four, five, six, seven, eight, nine or ten catalyst beds. In some embodiments, only one catalyst bed may be disposed in a single reactor.

In one embodiment, a variance (difference) between a height of each of the catalyst beds may be no more than 15%. In some embodiments, a variance (difference) between a height of each of the catalyst beds may be no more than 14%, 13%, 12% or 11%. In one embodiment, each of the catalyst beds independently may have a height of from 2.0 m to 2.8 m. The height of each of the catalyst beds may be obtained by measuring the vertical distance from the bottommost surface to the topmost surface of the catalyst bed. In some embodiments, each of the catalyst beds independently may have a height of from 2.1 m, 2.2 m, 2.3 m, 2.4 m, 2.5 m or 2.6 m, to 2.2 m, 2.3 m, 2.4 m, 2.5 m, 2.6 m, or 2.7 m. In one embodiment, the first catalyst bed may have a height of 2.0 m to 2.2 m. In some embodiments, the first catalyst bed may have a height of 2.1 m. In one embodiment, the third catalyst bed may have a height of 2.3 m to 2.6 m. In one embodiment, the third catalyst bed may have a height of 2.4 m or 2.5 m. In one embodiment, the first and second catalyst beds may have the same height. In one embodiment, the third and fourth catalyst beds may have a height different from the first and second catalyst bed. In one embodiment, each of the third and fourth catalyst beds may have a height that may be 10% to 50% greater than the height of the first catalyst bed. In some embodiments, each of the third and fourth catalyst beds has a height that may be 5%, 6%, 7%, 8%, 9%, 10%, 12%, 15%, 18%, 20%, 24%, 27%, 30%, 35%, 40% or 45% greater than the height of the first catalyst bed. In one embodiment, the fifth catalyst bed may have a height that is 10% to 50% greater than the height of the first catalyst bed. In some embodiments, the fifth catalyst bed has a height that may be 5%, 6%, 7%, 8%, 9%, 10%, 12%, 15%, 18%, 20%, 24%, 27%, 30%, 35%, 40% or 45% greater than the height of the first catalyst bed. In one embodiment, the fourth and fifth catalyst beds may have the same height.

The desired catalyst bed height may be calculated by selecting a desired catalyst campaign duration and the average feed of the first fluid to the reactors. A desirable catalyst campaign duration may be one during which the yield of the olefin may be maintained at a level that is the same as or at least 90%, 91%, 92%, 93%, 94%, 95%, 96%, 97%, 98%, or 99% of the yield of the olefin at the beginning of the catalyst campaign.

In one embodiment, the fourth and fifth catalyst beds may have the same volume. In some embodiments, the fourth and fifth catalyst beds each has a volume that may be from 20 to 23 m³ or from 22 to 23 m³. In one embodiment, a volume of the catalyst beds may increase from the first catalyst bed to the fourth catalyst bed. In some embodiments, a volume of each of the first to fourth catalyst beds may be from 7 to 9 m³, from 10 to 15 m³, from 16 to 19 m³, and from 20 to 23 m³, respectively. In some embodiments, a volume of each of the first to fourth catalyst beds may be from 8 to 9 m³, from 10 to 11 m³, from 17 to 18 m³, and from 22 to 23 m³, respectively.

### Reactors

In some embodiments, the reactors may be fixed bed reactors.

In one embodiment, at least one of the plurality of reactors may have a weight hourly space velocity of the alcohol of more than 0.6/h and up to 1.5/h per kg of catalyst. In some embodiments, at least one of the plurality of reactors may have a weight hourly space velocity of the alcohol of more than 0.7/h, 0.8/h, 0.9/h, 1/h, 1.1/h, 1.2/h, or 1.3/h, and/or up to 0.8/h, 0.9/h, 1.0/h, 1.1/h, 1.2/h, 1.3/h or 1.4/h per kg of catalyst. In one embodiment, at least one of the plurality of reactors may have a weight hourly space velocity of the alcohol of more than 1/h and up to 1.3/h per kg of catalyst.

In some embodiments, there may be three, four, five, six, seven, eight, nine or ten reactors. In one embodiment, the plurality of reactors may include a first group of reactors including reactors each having a first height, and a second group of reactors including reactors each having a second height, where the first height may be different from the second height. In some embodiments, the first group of reactors may precede the second group of reactors, and the first height may be shorter than the second height.

In one embodiment, the plurality of reactors may include, in order, a first reactor including a first catalyst bed, a second reactor including a second catalyst bed, a third reactor including a third catalyst bed, and a fourth reactor including a fourth catalyst bed. In one embodiment, the plurality of reactors further may include, after the fourth reactor, a fifth reactor including a fifth catalyst bed.

In one embodiment, a variance (difference) between a height of each of the plurality of reactors may be no more than 15%. In some embodiments, a variance (difference) between a height of each of the plurality of reactors may be no more than 14%, 13%, 12% or 11%. In one embodiment, a variance (difference) between a height of each of the plurality of reactors may be no more than 10%. In one embodiment, each of the plurality of reactors independently may have a height of from 2.5 m to 4.0 m. The height of each reactor may be obtained by measuring the vertical distance from the bottommost surface to the topmost surface of the reactor. In some embodiments, each of the plurality of reactors independently may have a height of from 2.6 m, 2.7 m, 2.8 m, 2.9 m, 3 m, 3.1 m, 3.2 m, 3.3 m, 3.4 m, 3.5 m, 3.6 m, 3.7 m, or 3.8 m and up to 2.7 m, 2.8 m, 2.9 m, 3 m, 3.1 m, 3.2 m, 3.3 m, 3.4 m, 3.5 m, 3.6 m, 3.7 m or 3.8 m.

In one embodiment, the first reactor may have a height of 2.8 to 3.1 m. In some embodiments, the first reactor may have a height of 2.9 m or 3 m. In one embodiment, the third reactor may have a height of 3.2 to 3.5 m. In some embodiments, the third reactor may have a height of 3.3 m or 3.4 m. In one embodiment, the first and second reactors may have the same height. In one embodiment, the first and second reactors may each have a height of 3 m. In one embodiment, the remaining reactors may have a height different from the first and second reactors. In one embodiment, each of the third and fourth reactors has a height that may be 10% to 50% greater than the height of the first reactor. In some embodiments, each of the third and fourth reactors has a height that may be 5%, 6%, 7%, 8%, 9%, 10%, 12%, 15%, 18%, 20%, 24%, 27%, 30%, 35%, 40% or 45% greater than the height of the first reactor. In one embodiment, the fifth reactor may have a height that is 10% to 50% greater than the height of the first reactor. In some embodiments, the fifth reactor has a height that may be 5%, 6%, 7%, 8%, 9%, 10%, 12%, 15%, 18%, 20%, 24%, 27%, 30%, 35%, 40% or 45% greater than the height of the first reactor. In one embodiment, the third to fifth reactors may have the same height. In some embodiments, the third to fifth reactors may each have a height of 3.3 m.

In one embodiment, the fourth and fifth reactors may have the same volume. In some embodiments, the fourth and fifth reactors each has a volume that may be from 30 to 33 m³ or from 31 to 32 m³. In one embodiment, a volume of each of the first to fourth reactors may increase from the first reactor to the fourth reactor. In some embodiments, a volume of each of the first to fourth reactors may be from 10 to 13 m³, from 14 to 20 m³, from 21 to 29 m³, and from 30 to 33 m³, respectively. In some embodiments, a volume of each of the first to fourth reactors may be from 11 to 12 m³, from 15 to 16 m³, from 24 to 25 m³, and from 31 to 32 m³, respectively.

In one embodiment, at least one of the plurality of reactors may be a spare reactor. In one embodiment, the spare reactor may have the same height and/or volume as another reactor among the plurality of reactors. In one embodiment, the spare reactor may have the same height and/or volume as another reactor immediately preceding the spare reactor. In one embodiment, the plurality of the reactors may include five reactors. In one embodiment, only one of the five reactors may be a spare reactor. In some embodiments, the spare reactor is the fifth reactor. In one embodiment, the spare reactor may be configured to be a spare for any of the other reactors among the plurality of reactors. In one embodiment, the spare reactor may be the last reactor among the plurality of the reactors. In one embodiment, the spare reactor may have the greatest height and/or volume among reactors in the plurality of the reactors. In some embodiments, the spare reactor may be the same as or similar to the other reactors in terms of the catalyst campaign duration (as described further below) and the amount of time for the regeneration or replacement of the catalyst bed (as described further below).

In some embodiments, the plurality of reactors may be arranged in a series-parallel arrangement. Compared to a series arrangement, the series-parallel combination may provide the benefit of facilitating the maintenance of a high temperature inside the reactors since the heated first fluid (e.g., ethanol) may be added in each reactor. Besides this, it may improve selectivity since adding fresh alcohol may avoid more by-products. For example, when the heated first fluid is ethanol, ethylene may be converted to other molecules when the reactors are arranged in series. Furthermore, when compared to a parallel arrangement, the series-parallel combination may reduce steam consumption since this consumption may be based only on the first reactor conditions.

In some embodiments, any or all of the reactors among the plurality of reactors may be in a parallel arrangement, and any or all of the reactors among the plurality of reactors may be in a series arrangement. In some embodiments, the first two, three, four, five or six reactors, the last two, three, four, five or six reactors, or any two, three, four, five or six reactors among the plurality of reactors may be in a parallel arrangement. In some embodiments, the first two, three, four, five or six reactors, the last two, three, four, five or six reactors, or any two, three, four, five or six reactors among the plurality of reactors may be in a series arrangement. In some embodiments, all of the reactors may be connected in parallel and in series with each other. In some embodiments, all of the reactors may be in a parallel arrangement.

In some embodiments, there may be a first group of reactors and a second group of reactors. In some embodiments, the reactors within the first group may be connected in parallel and in series with each other and at least one of the reactors within the second group, and the reactors within the second group may be in series with each other. In some embodiments, the reactors within the second group may be connected in parallel and in series with each other and at least one of the reactors within the first group, and the reactors within the first group may be in series with each other. In some embodiments, the reactors within the first and second groups may be connected in parallel and in series with each other.

In some embodiments, there may be five reactors. In some embodiments, the first and second reactors may be connected in parallel and in series with each other, and the second to fifth reactors may be connected in series. In some embodiments, the first to third reactors may be connected in parallel and in series with each other, and the third to fifth reactors may be connected in series. In some embodiments, the first to fourth reactors may be connected in parallel and in series with each other, and the fifth reactor may be connected in series with the fourth reactor. In some embodiments, the fourth and fifth reactors may be connected in parallel and in series to each other, and the first to fourth reactors may be connected in series. In some embodiments, the third to fifth reactors may be connected in parallel and in series with each other, and the first to third reactors may be connected in series. In some embodiments, the second to fifth reactors may be connected in parallel and in series with each other, and the second reactor may be connected in series with the first reactor.

### Furnace-Reactor Couple

In one embodiment, at least one of the plurality of reactors may be fluidly coupled to a furnace to form a furnace-reactor couple. In one embodiment, the method may further include passing the first fluid through the furnace before the first fluid enters the reactor in the same furnace-reactor couple. In one embodiment, the furnace may be directly coupled to the reactor. In one embodiment, the furnace-reactor couple may include a single furnace and a single reactor. In one embodiment, a plurality of the furnace-reactor couples may be present, and each of the furnace-reactor couples may be fluidly coupled to each other. In some embodiments, the reactor in a furnace-reactor couple may be directly coupled to the furnace in the immediately following furnace-reactor couple.

In some embodiments, there may be three, four, five, six, seven, eight, nine or ten furnace-reactor couples. In one embodiment, the plurality of furnace-reactor couples may include a first group of furnace-reactor couples including reactors each having a first height, and a second group of furnace-reactor couples including reactors each having a second height, where the first height may be different from the second height. In some embodiments, the first group of furnace-reactor couples may precede the second group of reactors, and the first height may be shorter than the second height.

In one embodiment, the plurality of furnace-reactor couples may include, in order, a first furnace-reactor couple including a first furnace and a first reactor including a first catalyst bed, a second furnace-reactor couple including a second furnace and a second reactor including a second catalyst bed, a third furnace-reactor including a third furnace and as third reactor including a third catalyst bed, and a fourth furnace-reactor couple including a fourth furnace and a fourth reactor including a fourth catalyst bed. In one embodiment, the plurality of reactors further may include, after the fourth furnace-reactor couple, a fifth furnace-reactor couple including a fifth furnace and a fifth reactor including a fifth catalyst bed.

In one embodiment, at least one of the plurality of the furnace-reactor couples may be a spare furnace-reactor couple. In one embodiment, the spare furnace-reactor couple may include a reactor that may have the same height and/or volume as another reactor in another furnace-reactor couple among the plurality of the furnace-reactor couples. In one embodiment, the spare furnace-reactor couple may include a reactor that may have the same height and/or volume as another reactor in another furnace-reactor couple immediately preceding the spare furnace-reactor couple. In one embodiment, the plurality of the furnace-reactor couples may include five furnace-reactor couples. In one embodiment, only one of the five of the furnace-reactor couples may be a spare furnace-reactor couple. In some embodiments, the spare furnace-reactor couple includes the fifth reactor only among the plurality of reactors. In one embodiment, the spare furnace-reactor couple may be configured to be a spare for any of the other furnace-reactor couples among the plurality of furnace-reactor couples. In one embodiment, the spare furnace-reactor couple may be the last furnace-reactor couple among the plurality of the furnace-reactor couples. In one embodiment, the reactor in the spare furnace-reactor couple may have the greatest height and/or volume among reactors in the plurality of the furnace-reactor couples. In some embodiments, the spare furnace-reactor couple may be the same as or similar to the other furnace-reactor couples in terms of the catalyst campaign duration and the amount of time for the regeneration or replacement of the catalyst bed.

In one embodiment, the plurality of furnace-reactor couples may be in a series-parallel arrangement in which any or all of the furnace-reactor couples may be series-connected and any or all of the furnace-reactor couples may be parallel to each other. In some embodiments, the furnace-reactor couples in a parallel arrangement may be the first two, three, four, five or six furnace-reactor couples, the last two, three, four, five or six furnace-reactor couples, or any two, three, four, five or six furnace-reactor couples among the plurality of furnace-reactor couples. In some embodiments, the first two, three, four, five or six furnace-reactor couples, the last two, three, four, five or six furnace-reactor couples, or any two, three, four, five or six furnace-reactor couples among the plurality of reactors may be in a series arrangement. In some embodiments, all of the furnace-reactor couples may be connected in parallel and in series with each other.

In some embodiments, there may be a first group of furnace-reactor couples and a second group of furnace-reactor couples. In some embodiments, the furnace-reactor couples within the first group may be connected in parallel and in series with each other and at least one of the furnace-reactor couples within the second group, and the furnace-reactor couples within the second group may be in series with each other. In some embodiments, the furnace-reactor couples within the second group may be connected in parallel and in series with each other and at least one of the furnace-reactor couples within the first group, and the furnace-reactor couples within the first group may be in series with each other. In some embodiments, the furnace-reactor couples within the first and second groups may be connected in parallel and in series with each other.

In some embodiments, there may be five furnace-reactor couples. In some embodiments, the first and second furnace-reactor couples may be connected in parallel and in series to each other, and the second to fifth furnace-reactor couples may be connected in series. In some embodiments, the first to third furnace-reactor couples may be connected in parallel and in series with each other, and the third to fifth furnace-reactor couples may be connected in series. In some embodiments, the first to fourth furnace-reactor couples may be connected in parallel and in series with each other, and the fifth furnace-reactor couple may be connected in series with the fourth furnace-reactor couple. In some embodiments, the fourth and fifth furnace-reactor couples may be connected in parallel and in series to each other, and the first to fourth furnace-reactor couples may be connected in series. In some embodiments, the third to fifth furnace-reactor couples may be connected in parallel and in series with each other, and the first to third furnace-reactor couples may be connected in series. In some embodiments, the second to fifth furnace-reactor couples may be connected in parallel and in series.

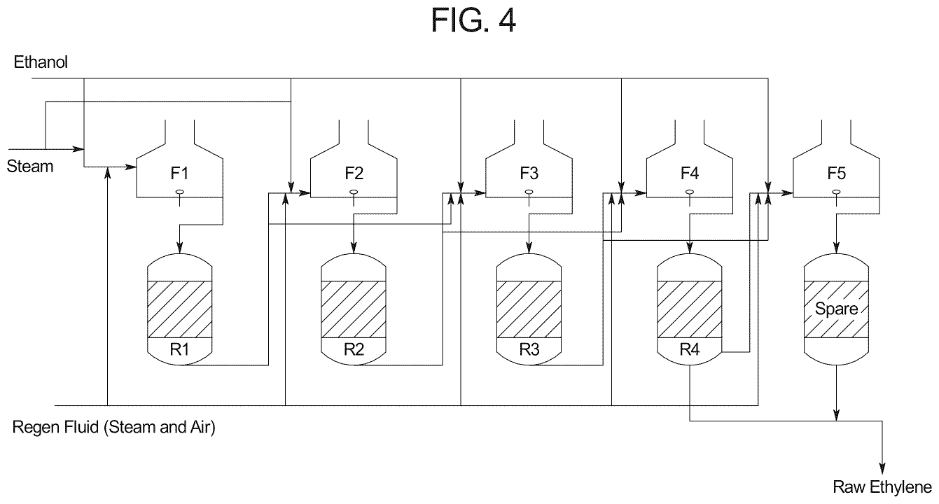

In some embodiments, all of the furnace-reactor couples may be connected in parallel and in series with each other. In some embodiments, the plurality of furnace-reactor couples is arranged in a series-parallel arrangement as illustrated in FIG. 4. The terms “R1”, “R2”, “R3”, “R4”, and “Spare” in FIG. 4 are intended to mean the first, second, third, fourth, and fifth reactors, respectively, and the terms “F1”, “F2”, “F3”, “F4”, and “F5” are intended to mean the first, second, third, fourth, and fifth furnaces, respectively.

### Catalyst Campaign

In one embodiment, the catalyst campaign may be between five and seven months. In some embodiments, the catalyst campaign may be from three, four, five, six, seven or eight months to four, five, six, seven, eight, nine or ten months. In some embodiments, the catalyst campaign may be between six and seven months. In one embodiment, the catalyst campaign may be six months.

In some embodiments, the catalyst campaign for each reactor may overlap with each other. This means that all of the reactors are operating. In some embodiments, the catalyst campaign for each reactor may overlap for at least three, four or five months, and/or up to four, five, six or seven months. In some embodiments, the catalyst campaign for each reactor may overlap for four to five months. In one embodiment, all of the plurality of reactors may operate together at 60% to 80% of capacity. In some embodiments, all of the plurality of reactors may operate together at least 61%, 62%, 63%, 64%, 65%, 66%, 67%, 68%, 69%, 70%, 71%, 72%, 73%, 74%, 75%, 76%, 77%, 78% or 79%, and/or up to 62%, 63%, 64%, 65%, 66%, 67%, 68%, 69%, 70%, 71%, 72%, 73%, 74%, 75%, 76%, 77%, 78%, 79% or 80% of capacity. In one embodiment, all of the plurality of the furnace-reactor couples may operate together at 60% to 80% of capacity. In some embodiments, all of the plurality of the furnace-reactor couples may operate together at least 61%, 62%, 63%, 64%, 65%, 66%, 67%, 68%, 69%, 70%, 71%, 72%, 73%, 74%, 75%, 76%, 77%, 78% or 79%, and/or up to 62%, 63%, 64%, 65%, 66%, 67%, 68%, 69%, 70%, 71%, 72%, 73%, 74%, 75%, 76%, 77%, 78%, 79% or 80% of capacity.

### Regeneration

In one embodiment, the method may further include, after the catalyst campaign, decoupling at least one of the plurality of reactors from the remaining reactors, and regenerating the catalyst bed or changing the catalyst bed in the at least one decoupled reactor. The decoupled reactor(s) may not be used to dehydrate the alcohol to form the olefin. In one embodiment, the decoupling may occur immediately after the catalyst campaign. In one embodiment, the remaining reactors may operate together at 100% of capacity. In some embodiments, the remaining reactors may operate together at more than 80%, 81%, 82%, 83%, 84%, 85%, 86%, 87%, 88%, 89%, 90%, 91%, 92%, 93%, 94%, 95%, 96%, 97%, 98% or 99% of capacity.

The regenerating of the catalyst bed may include passing at least steam and air through the catalyst bed in the decoupled furnace-reactor couple. The regenerating of the catalyst bed may take from five, six, seven, eight or nine days and up to seven, eight, nine, ten, twelve, fifteen or twenty days. In some embodiments, the regenerating of the catalyst bed may take ten days. The changing of the catalyst bed may take from five, six, seven, eight or nine days and up to seven, eight, nine, ten, twelve, fifteen or twenty days. In some embodiments, the changing of the catalyst bed may take ten days.

In some embodiments, the method may further include coupling the decoupled reactor having the regenerated catalyst bed or replaced catalyst bed to the remaining reactors, and followed by decoupling at least one other reactor so as to regenerate or change its catalyst bed.

In one embodiment, the method may further include, after the catalyst campaign, decoupling at least one of the plurality of the furnace-reactor couples from the remaining furnace-reactor couples, and regenerating the catalyst bed or changing the catalyst bed in the at least one decoupled furnace-reactor couple. The decoupled furnace-reactor couple(s) may not be used to dehydrate the alcohol to form the olefin. In one embodiment, the decoupling may occur immediately after the catalyst campaign.

In one embodiment, the remaining furnace-reactor couples may operate together at 100% of capacity. In some embodiments, the remaining furnace-reactor couples may operate together at more than 80%, 81%, 82%, 83%, 84%, 85%, 86%, 87%, 88%, 89%, 90%, 91%, 92%, 93%, 94%, 95%, 96%, 97%, 98% or 99% of capacity. In a comparative process operating with four reactors and four furnaces, a reactor being regenerated would be by-passed, reducing the production capacity significantly. The spare furnace-reactor couple as disclosed herein may maintain the production at full (e.g., 100%) capacity all the time, even during the regeneration or replacement of other catalyst bed(s). The spare furnace-reactor couple may also enhance the yield of the process by allowing frequent regeneration to reduce by-products formation.

In some embodiments, the method may further include coupling the decoupled furnace-reactor couple having the regenerated catalyst bed or replaced catalyst bed to the remaining furnace-reactor couples, and followed by decoupling at least one other furnace-reactor couple so as to regenerate or change its catalyst bed. The coupling and decoupling processes may be repeated as necessary so that all of the catalyst beds may be regenerated or replaced to, for example, maintain a desirable product yield.

### Alcohol

In some embodiments, the alcohol may be a precursor to an olefin that may be industrially important. For example, the olefin may be a raw material for chemicals and/or polymers.

In some embodiments, the alcohol may originate from sugarcane, corn, beetroot, and/or biomass. In one embodiment, the alcohol may include at least one of a C₂ alcohol, a C₃ alcohol, and a C₄ alcohol. In some embodiments, the alcohol may be a C₂ alcohol. In some embodiments, the alcohol may be a C₃ alcohol. In some embodiments, the alcohol may be a C₄ alcohol. In some embodiments, the alcohol may be a C₅ alcohol, a C₆ alcohol, a C₇ alcohol, and/or a C₈ alcohol.

In some embodiments, the alcohol may be a C₂ alcohol, and the olefin may be a C₂ alkene. In some embodiments, the alcohol may be a C₃ alcohol, and the olefin may be a C₃ alkene. In some embodiments, the alcohol may be a C₄ alcohol, and the olefin may be a C₄ alkene.

### Olefin

In one embodiment, the olefin may include an alkene having the same number of carbons atoms as the alcohol. In some embodiments, the alcohol may be a C₂ alcohol, and the olefin is a C₂ alkene. In some embodiments, the alkene may be a C₂ alkene, for example, ethylene. In some embodiments, the alkene may be a C₃ alkene. In some embodiments, the alkene may be a C₄ alkene.

System for Converting an Alcohol into an Olefin

In one aspect, the present disclosure provides a system including a plurality of reactors, each reactor may include a catalyst bed and configured to convert an alcohol into an olefin. The plurality of reactors is fluidly coupled to each other, and at least one of the catalyst beds is configured to have a catalytic-active zone that is at least 90% of the catalyst bed during a catalyst campaign. In some embodiments, the corresponding catalyst bed may include a catalytic-active zone that is at least 91%, 92%, 93%, 94%, 95%, 96%, 97%, 98%, or 99% of the catalyst bed. In one embodiment, the catalytic-active zone may be 100% of the catalyst bed during the catalyst campaign. In one embodiment, at least one of the catalyst beds may further include an idle catalyst zone that is less than 10% of the catalyst bed during the catalyst campaign. In some embodiments, the idle catalyst zone that may be less than 9%, 8%, 7%, 6%, 5%, 4%, 3%, 2%, or 1% of the catalyst bed during the catalyst campaign. In one embodiment, at least one of the catalyst beds may not include an idle catalyst zone during the catalyst campaign.

### EXAMPLES

### Example 1. Determining the Desired Catalyst Bed Height and Other Reactor Characteristics

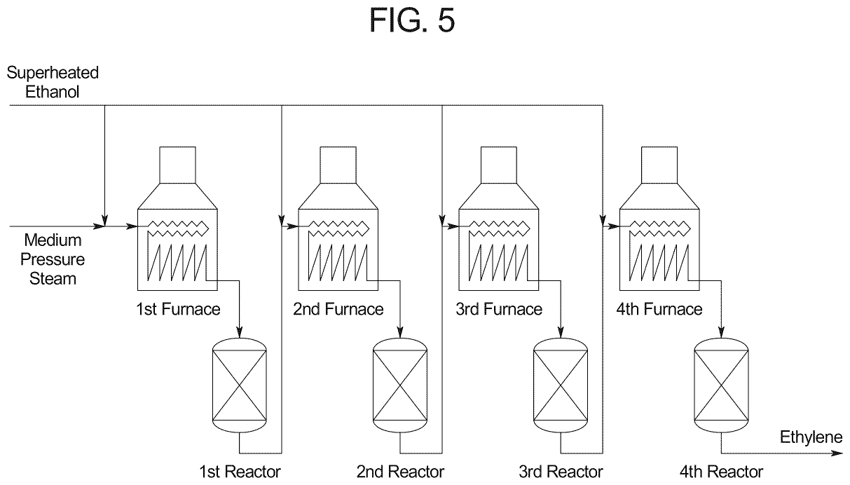

Ethylene production occurs by ethanol dehydration. This process may be implemented through four reactors and four furnaces, as illustrated FIG. 5, which shows a comparative system that does not have a spare reactor and the catalytic-active zone is less than 90% of the catalyst bed. The ethanol dehydration reactors in the comparative process are fixed bed reactors, which have the inerts at the top, the catalyst bed in the middle, and the inerts at the bottom of the reactors. Along the reactors' height, there are several temperature transmitters, which monitor the temperature profile inside the reactors to understand where the reaction front is. The space between transmitters is called layers, which can be inert layers or catalyst layers.

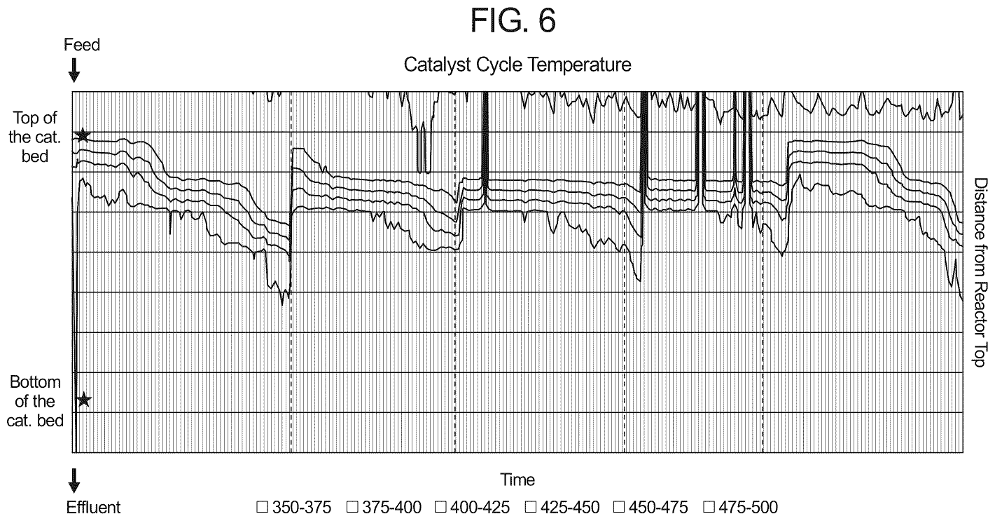

To calculate the catalyst bed height, a relevant concept is the reaction front section. The reaction front is defined as the location along the catalyst bed where most of the chemical reaction occurs in a given moment. As the reaction progresses, the reaction front slowly moves downwards, as illustrated in FIG. 6.

Theoretically, the reaction front should reach the bottom of the reactor at the end of the catalyst campaign. After that, the catalyst will be regenerated, and the new catalyst campaign begins.

One possible reason for the by-product formation may be the temperature in the buffer section, as illustrated in FIG. 6. Low temperature (e.g., about 350° C.) may favor the production of by-products, particularly ethane. The early catalyst regeneration indicates that the bed height is overdesigned. An overdesigned reactor provides a buffer section longer than it should be and, therefore, by-product formation is higher than it could be. Long bed heights may lead to low temperatures in the idle catalyst layers in the buffer section, which leads to unnecessary by-product formation. The overall yield and selectivity may be reduced.

The present application relates to determining the desired bed height, which may be longer than the minimum reaction front section but shorter than the bed height of reactors in the comparative process. Another aspect of the present application relates to reducing by-product formation as compared to the reactors in the comparative process.

In the present application, the sizes of the reactors were determined individually. To define the catalyst bed height for each reactor, temperature is a relevant parameter since it is believed to be related to by-product formation in the buffer section. Therefore, understanding the temperature profile inside the reactors may be important for defining the bed height.

Table 1 lists the height variation of the reactors and weight hourly space velocity (WHSV) of ethanol for reactors in the comparative process for a certain capacity. Based on a study of the ethanol dehydration plant, a common behavior (e.g., a large number of idle layers) was observed in all the reactors.

To calculate the desired height of the catalyst bed, the average feed of ethanol to the reactor were chosen. Since ethanol feed rate was kept the same as the comparative process, smaller reactors disclosed herein resulted in higher weight hourly space velocity of ethanol, which indicates lower catalyst consumption, and hence, lower catalyst costs.

TABLE 1
Features of reactors in the comparative process

| | WHSV | | | | |
|---|---|---|---|---|---|
| Height (m) | 0.6 | 0.8 | 0.9 | 1 | 1.2 |
| Reactor 1/2 | 5.4 | 4.2 | 3.7 | 3.3 | 2.7 |
| Reactor 3/4 | 6.1 | 4.7 | 4.2 | 3.7 | 3.2 |

### Example 2. Reduced Catalyst Bed Height Leads to a Lower Pressure Drop

In the comparative process and the process according to an embodiment of the present disclosure, ethanol was pressurized and lost pressure through the equipment (including the reactors) and in the lines until it reached the suction of the charge compressor. A lower pressure drop along the catalyst bed would allow the stream to reach the suction of the charge compressor at a higher pressure. At a higher pressure, the compressor size can be reduced and, therefore, capital expenditure and power may be reduced. The compressor in the comparative process has three stages. Simulations have shown that with smaller reactors according to embodiments of the present disclosure, a one-stage compressor would be suitable for the purposes of the present disclosure.

A system curve for the comparative process was built to understand the losses of pressure in each equipment until the product reached the suction of the charge compressor. A direct correlation between pressure drop and bed height was observed. Based on the information, the system curve was recalculated for the process according to an embodiment of the present disclosure. For the recalculation, the fifth furnace-reactor couple was taken into account with 70% of the design flow in each reactor. This was the operation condition for 9 months. Also, there were additional equipment (the fifth furnace and fifth reactor) arranged in series until the suction of the compressor.

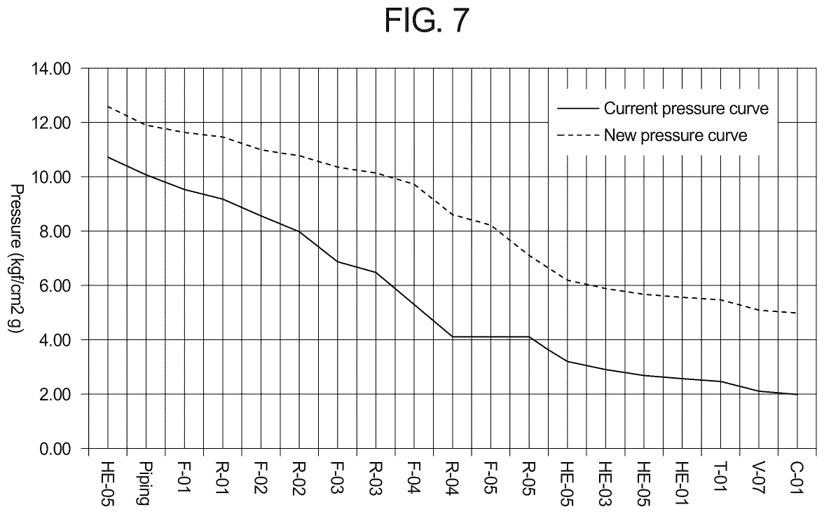

FIG. 7 presents the system curve according to a comparative process (“current pressure curve”) and a process according to an embodiment of the present disclosure (“new pressure curve”). In FIG. 7, for example, the terms “F-01” and “R-01” refer to the first furnace and reactor, respectively, “HE-05” refers to a heat exchanger, “T-01” refers to a tower, “V-07” refers to a vessel, and “C-01” refers to compressor. To reach 5 kgf/cm² (g) in the suction of the charge compressor in the process as disclosed in the present disclosure, the initial pressure of the system was also increased.

### Example 3. Investigating the Formation of by-Products

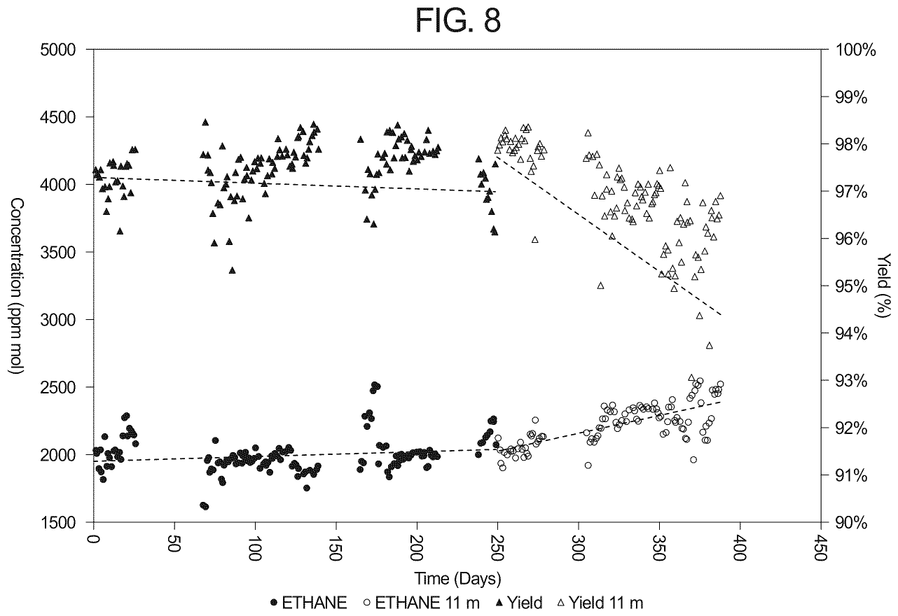

Ethane, one of the by-products of ethanol dehydration, is difficult to separate from ethylene and may affect directly the yield of ethylene. This study used the comparative system and considered the reaction as one system. The study focused on the time between the intervention in each reactor (regeneration or change of the catalyst). FIG. 8 shows a typical behavior of ethane and the yield of ethylene in a catalyst campaign of 11 months. Based on this behavior, a catalyst campaign of 6 months (period between regenerations or catalyst change) may allow for a higher yield and lower ethane concentration. This was the period considered in the following study.

To better understand how the 6-month campaign of the reactors according to the present disclosure will be implemented, an occupation diagram of the reactors was built. According to Table 2, 6 months of catalyst campaign for each reactor and 10 days of regeneration or catalyst change results in full 9 months in operation. In Table 2, “x” indicates the reactor is in operation, and the number is the number of days that the reactor is under regeneration.

TABLE 2
Occupation diagram of the reactors according
to the present disclosure over a year

| | Month | | | | | | | | | | | | | | | |
|---|---|---|---|---|---|---|---|---|---|---|---|---|---|---|---|---|
| Reactor | 1 | | | 2 | 3 | 4 | 5 | 6 | 7 | | | 8 | 9 | 10 | 11 | 12 |
| R1 | 10 | x | x | x | x | x | x | x | 10 | x | x | x | x | x | x | x |

| | | | | | | | | | | | | | | | | |
|---|---|---|---|---|---|---|---|---|---|---|---|---|---|---|---|---|
| R2 | x | x | x | x | x | x | x | 10 | x | x | x | x | x | x | x | 10 |
| R3 | x | x | x | x | x | x | 10 | x | x | x | x | x | x | x | 10 | x |
| R4 | x | x | x | x | x | 10 | x | x | x | x | x | x | x | x | x | x |

| | | | | | | | | | | | | | | | | |
|---|---|---|---|---|---|---|---|---|---|---|---|---|---|---|---|---|
| R5 | x | 10 | x | x | x | x | x | x | x | 10 | x | x | x | x | x | x |

In summary, working with smaller reactors led to the reduction of the compressor size. With this change and the addition of a fifth reactor and its furnace, the capital expenditures were reduced significantly, with catalyst consumption being reduced by over half. The system according to the present disclosure also consumed less ethanol, electric energy, and steam, and affected fuel gas consumption. These variations influence the CO₂ footprint, which was also reduced.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using consisting of and/or consisting essentially of language. Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims and their equivalents. All references, patents, and patent applications referred to in this application are herein incorporated by reference in their entirety.

1. A method of converting an alcohol into an olefin, the method comprising:
passing a first fluid comprising the alcohol through a plurality of reactors, each reactor comprising a catalyst bed,
wherein, during a catalyst campaign for at least one of the plurality of reactors, the corresponding catalyst bed includes a catalytic-active zone that is at least 90% of the catalyst bed.

2. The method of claim 1, wherein the catalytic-active zone is 100% of the catalyst bed during the catalyst campaign.

3. The method of claim 1, wherein at least one of the catalyst beds further includes an idle catalyst zone that is less than 10% of the catalyst bed during the catalyst campaign, and/or
wherein the catalytic-active zone operates at a temperature of 280° C. to 500° C.

4. The method of claim 1, wherein at least one of the catalyst beds does not include an idle catalyst zone during the catalyst campaign.

5-6. (canceled)

7. The method of claim 1,
wherein the alcohol comprises at least one of a C₂ alcohol, a C₃ alcohol, and a C₄ alcohol, and/or
wherein the olefin comprises an alkene having the same number of carbons atoms as the alcohol, and/or
wherein the olefin comprises ethylene, and the catalytic-active zone operates at a temperature of 360° C. to 470° C.

8-9. (canceled)

10. The method of claim 1, further comprising:
contacting the first fluid with a second fluid comprising steam prior to the first fluid contacting the plurality of reactors, wherein a ratio of the steam to the alcohol is from 1:1 to 3:1,
obtaining a third fluid from at least one of the plurality of reactors, wherein the third fluid comprises the alcohol at a concentration lower than a concentration of the alcohol in the first fluid,
contacting the third fluid with the first fluid prior to contacting the third fluid with another reactor among the plurality of reactors, and
obtaining from an end of the plurality of reactors, a fourth fluid comprising the olefin and an alkane that has the same number of carbon atoms, wherein an amount of the olefin in the fourth fluid is at least 1,000 ppm, mole basis, greater than an amount of the alkane.

11. The method of claim 10, wherein an amount of the alkane in the fourth fluid is 2,500 ppm or less.

12. The method of claim 10, further comprising pressurizing the fourth fluid with a one-stage compressor connected to an end of the plurality of reactors, and/or
wherein the method excludes pressurizing the fourth fluid with a multi-stage compressor.

13. (canceled)

14. The method of claim 10, further comprising feeding the first fluid to at least one of the plurality of reactors at a temperature more than 280° C.

15. The method of claim 14, wherein the temperature is more than 400° C.

16. (canceled)

17. The method of claim 1, wherein the plurality of reactors is in a series-parallel arrangement, and/or
wherein at least one of the plurality of reactors has a weight hourly space velocity of the alcohol of more than 0.6/h and up to 1.5/h.

18-19. (canceled)

20. The method of claim 1, wherein the plurality of reactors comprises:
a first group of reactors including reactors each having a first height, and
a second group of reactors including reactors each having a second height,
wherein the first height is different from the second height.

21. The method of claim 1, wherein the plurality of reactors comprises, in order:
a first reactor comprising a first catalyst bed,
a second reactor comprising a second catalyst bed,
a third reactor comprising a third catalyst bed, and
a fourth reactor comprising a fourth catalyst bed.

22. The method of claim 21, wherein the plurality of reactors further comprises, after the fourth reactor, a fifth reactor comprising a fifth catalyst bed, and/or
wherein a variance between a height of each of the plurality of reactors is no more than 15%.

23. (canceled)

24. The method of claim 22, wherein a variance between a height of each of the plurality of reactors is no more than 10%, and/or
wherein each of the plurality of reactors independently has a height of from 2.5 m to 4.0 m, and/or
wherein the first reactor has a height of 2.8 to 3.1 m, and/or
wherein the third reactor has a height of 3.2 to 3.5 m, and/or
wherein the first and second reactors have the same height, and/or
wherein the remaining reactors have a height different from the first and second reactors, and/or
wherein each of the third and fourth reactors has a height that is 10% to 50% greater than the height of the first reactor, and/or
wherein the fifth reactor has a height that is 10% to 50% greater than the height of the first reactor, and/or
wherein the third to fifth reactors have the same height.

25-32. (canceled)

33. The method of claim 22, wherein the fourth and fifth reactors have the same volume, and/or
wherein a volume of each of the first to fourth reactors increases from the first reactor to the fourth reactor.

34. (canceled)

35. The method of claim 22, wherein a variance between a height of each of the catalyst beds is no more than 15%, and/or
wherein each of the catalyst beds independently has a height of from 2.0 m to 2.8 m, and/or
wherein the first catalyst bed has a height of 2.0 m to 2.2 m, and/or
wherein the third catalyst bed has a height of 2.3 m to 2.6 m, and/or
wherein the first and second catalyst beds have the same height, and/or
wherein the third and fourth catalyst beds have a height different from the first and second catalyst bed, and/or
wherein each of the third and fourth catalyst beds has a height that is 10% to 50% greater than the height of the first catalyst bed, and/or
wherein the fifth catalyst bed has a height that is 10% to 50% greater than the height of the first catalyst bed, and/or
wherein the fourth and fifth catalyst beds have the same height.

36-43. (canceled)

44. The method of claim 22, wherein the fourth and fifth catalyst beds have the same volume, and/or
wherein a volume of the catalyst beds increases from the first catalyst bed to the fourth catalyst bed.

45. (canceled)

46. The method of claim 1, wherein at least one of the plurality of reactors is fluidly coupled to a furnace to form a furnace-reactor couple.

47. The method of claim 46, further comprising passing the first fluid through the furnace before the first fluid enters the reactor in the same furnace-reactor couple.

48. The method of claim 46, wherein the furnace-reactor couple comprises a single furnace and a single reactor, a plurality of the furnace-reactor couples is present, each of the furnace-reactor couples is fluidly coupled to each other, and at least one of the plurality of the furnace-reactor couples is a spare furnace-reactor couple.

49-50. (canceled)

51. The method of claim 48, wherein the spare furnace-reactor couple includes a reactor that has the same height and/or volume as another reactor in another furnace-reactor couple among the plurality of the furnace-reactor couples, and/or
wherein the spare furnace-reactor couple includes a reactor that has the same height and/or volume as another reactor in another furnace-reactor couple immediately preceding the spare furnace-reactor couple, and/or
wherein the plurality of the furnace-reactor couples includes five furnace-reactor couples, and only one of the five of the furnace-reactor couples is a spare furnace-reactor couple, and/or
wherein the spare furnace-reactor couple is configured to be a spare for any of the other furnace-reactor couples among the plurality of the furnace-reactor couples, and/or
wherein the spare furnace-reactor couple is the last furnace-reactor couple among the plurality of the furnace-reactor couples, and/or
wherein the reactor in the spare furnace-reactor couple has the greatest height and/or volume among reactors in the plurality of the furnace-reactor couples.

52-57. (canceled)

58. The method of claim 1, wherein the catalyst campaign is between five and seven months, and/or
wherein all of the plurality of the furnace-reactor couples operate together at 60% to 80% of capacity.

59. (canceled)

60. The method of claim 58, further comprising, after the catalyst campaign:
decoupling at least one of the plurality of the furnace-reactor couples from the remaining furnace-reactor couples, and
regenerating the catalyst bed or changing the catalyst bed in the at least one decoupled furnace-reactor couple.

61. The method of claim 60, wherein the decoupling occurs immediately after the catalyst campaign, and/or
wherein the remaining furnace-reactor couples operate together at 100% of capacity.

62-63. (canceled)

64. A system comprising:
a plurality of reactors, each reactor comprising a catalyst bed and configured to convert an alcohol into an olefin, wherein:
the plurality of reactors is fluidly coupled to each other, and
at least one of the catalyst beds is configured to have a catalytic-active zone that is at least 90% of the catalyst bed during a catalyst campaign.

65-98. (canceled)
